(19) United States
(12) Patent Application Publication
(10) Pub. No.: US 2022/0304604 A1
(43) Pub. Date: Sep. 29, 2022

(54) METHOD, DEVICE AND SYSTEM FOR TREATMENT OF ADHD

(71) Applicant: Q2Behave LLC, Providence, RI (US)

(72) Inventor: Geanina Oana Costea, Providence, RI (US)

(73) Assignee: Q2Behave LLC, Providence, RI (US)

(21) Appl. No.: 17/696,022

(22) Filed: Mar. 16, 2022

Related U.S. Application Data
(60) Provisional application No. 63/164,667, filed on Mar. 23, 2021.

(51) Int. Cl. A61B 5/16; A61B 5/00; G06F 1/16

(52) U.S. Cl. CPC A61B 5/168; A61B 5/165; A61B 5/4815; G06F 1/163

(57) ABSTRACT

A system used by a child diagnosed with ADHD and including a wearable device equipped with motion sensors and optionally a location or GPS tracker as a safety feature and using cloud based technology to facilitate the transfer of information among users. Motion data is used for two main purposes: 1) to objectively measure hyperactivity levels on specific metrics and optionally to detect presence or absence of aggression; and 2) to track the child's sleep-wake patterns (given a high percentage of children with ADHD are known to have sleep disorders). Behavior feedback is provided in real time based on the data obtained. Additional sensors may also be used to aid the assessment of child's behavioral functioning and sleep-wake patterns.

### CROSS REFERENCE TO RELATED APPLICATION

This application claims priority from earlier filed Provisional Application No. 63/164,667, filed Mar. 23, 2021, entitled “METHOD, DEVICE AND SYSTEM FOR TREATMENT OF ADHD,” incorporated by reference in its entirety, herein.

### BACKGROUND OF THE DISCLOSURE

The present disclosure relates generally to the field of Child Psychiatry (Pediatric Mental Health). More specifically, the present disclosure relates to a method and system for treatment intervention of Attention Deficit Hyperactivity Disorder (ADHD) in children, at least children 5-11 years of age. Further, the present disclosure relates to a treatment intervention that is used under professional guidance and, as indicated at times by the clinical picture, could be used along with medication interventions. The present disclosure relates to at least the targeting of two types of ADHD: Combined type (Hyperactive/Impulsive and Inattentive) and Hyperactive-Impulsive type and possibly the targeting of the ADHD Inattentive type. The method, device and system of the present disclosure relates to at least the treatment of mild and moderate cases of ADHD.

In the field of Child Psychiatry (Pediatric Mental Health), it is well known that the treatment of ADHD is very challenging and difficult. Notably, there is limited availability of services for pediatric mental health as well as an overburdened healthcare system causing long wait times for services. Thus, unfortunately, a high percentage of children that have mental health issues do not receive treatment. As a result, their condition worsens resulting in emergency room visits and hospitalizations. For example, as many 8.4% of children have ADHD, which raises a high level of concern.

It is well known in the field that ADHD has many significant health and economic adverse effects. For example, ADHD leads to significant functional impairment for the child which impacts peer and family relationships, as well as academic functioning. Behavior therapy and medications are the two most well-known evidence-based treatments for ADHD.

Behavior parent training is a form of behavior therapy. In current practice, parents are trained by a behavioral therapist, such as during an office visit, in the implementation of behavioral interventions including learning skills on how to respond and manage their child's behaviors. The principles of behavioral therapy focus on increasing desired behaviors by providing positive reinforcement. The behavior parent training has the following features: 1) Entails training parents in implementation of behavioral interventions (i.e. Home Token Economy), 2) Augmented with classroom-based intervention to facilitate skill generalization (i.e. Daily Report Card), 3) Grounded in contingency theory, 4) Used in the following clinical circumstances: a) Mild symptoms with minimal functional impairment, b) Uncertain diagnosis, c) Parents opting against medications, d) Outcomes optimization when combined with medications, e) Possibly lowering of medication dosage.

Therefore, a method, device and system addressing the key goals of behavior parent training, should support attending to child's misbehavior while optimizing the achievement of positive behavior. Such a system is directed towards automating the implementation of Home Token Economy and using a School Daily Report Card while indirectly facilitating the achievement of other goals for behavior parent training such as managing non-compliant behavior when in public and anticipating future misconduct.

Potential benefits of behavior parent training include long-lasting benefits of self-control and empowerment for the child, improved parent child relationship and decreased parental stress. However, in the current practice of behavior parent training there are challenges, such as outcomes being setting specific to where the intervention was implemented (e.g. home). Furthermore, while benefits can persist for at least several months, booster treatment may be necessary, requiring significant time/effort from caregivers. This highlights a crucial need for feasible/cost effective methods to implement behavioral treatment.

A Home Token Economy, as illustrated in the behavior chart of FIG. 1, for example, entails identifying up to 5 desired behaviors and rewards to incentivize the child to accomplish them. Of note, the desired behaviors are identified by the parent in collaboration with the behavioral therapist during the office visit.

It is also well known that teachers are also trained in behavioral interventions to be used in the classroom with the same goals as outlined above. For example, a Daily Report Card, as seen in FIG. 2, is used to mark child's performance on certain desired behaviors and then sent home in order for the parent to provide the child with a reward based on his/her performance. In conjunction with the Daily Report Card, a weekly schedule chart is also typically maintained as shown in FIG. 3.

However, there are many challenges of the current practice of Behavior Parent Training for treating ADHD. For example, it is difficult to sustain by parents or teachers because a) benefits cannot be seen for weeks or months of treatment, b) benefits can persist for several months but booster sessions may be needed, c) the rewards have to be immediate and consistent in order to be effective, d) parental stress/parent's own ADHD or other emotional needs could impact sustainability/outcome.

Also, outcomes, such as improved behavior, are typically setting specific. That is, if behavioral interventions are implemented only in the home, the changes will only typically occur at home and do not generalize to school, which is why the interventions must occur both in school and at home or other settings where behaviors are problematic. A further challenge is the cost in terms of time and effort to train and monitor parents/teachers and child response to interventions. In other words, the cost can be substantial for therapists, psychiatrists, behavioral programs, summer treatment camps, parent training, and the like. Also, despite the typical psychotherapy reimbursement rate for insurance, the costs of care are still substantial.

There is also reasonable probability that the rewards will not be provided immediately or consistently given competing factors for parent/teacher attention. Therefore, the intervention effectiveness is decreased, and the duration of treatment extended thereby requiring a higher utilization of services. Additionally, the rewards provided by parents and teachers are based on observation/subjective assessment and are binary (i.e. behavior is present or absent) with limited ability to reward small improvements in behavior. Moreover, parents often prefer behavior therapy to medications and additionally behavior therapy is the recommended first line of treatment for preschoolers partly due to their typical poor tolerance for medications.

There have been some attempts to address the foregoing known problems of treatment of ADHD.

For example, prior art efforts include an FDA approved digital therapeutic for the treatment of inattention in ADHD based on gaming. Such treatments are not standalone and are meant to be used along with traditional ADHD treatments. However, such treatments do not address the behavioral/hyperactivity component of ADHD.

Also, motion data has been in used for ADHD but as a diagnostic tool. Such method provides an objective measure of attention, impulsivity, and activity but it is not meant to be a standalone diagnostic tool and requires professional input. Moreover, such method is very expensive.

Wearable fitness and activity tracking devices for children are also well known, however, these are consumer products with no suitable professional use.

Therefore, there is a need for method, device and system for treating ADHD that is effective, efficient and cost-effective.

Therefore, there also a need to partially automate and optimize Behavior Parent Training which is a form of Behavior Therapy.

There is a further need for a method, device and system that is precise and objective.

Therefore, there is yet a further need for a method, device and system that uses motion data to objectively measure treatment targets (i.e. desired behavior).

There is yet a further need for a method, device and system that provides immediate feedback as positive reinforcement for desired behavior.

There is another need for a method, device and system that fosters generalization through the reinforcement of behaviors across settings, such as home and school.

There is a further need for a method, device and system to decrease parental stress in managing a child's ADHD.

There is yet a further need to provide a method, device and system that improves parent-child interactions.

Further, there is a need to reduce the amount of medication given to a child while still treating their ADHD.

Moreover, there is a need to increase the cost effectiveness of treatment while improving the outcome and reducing the need for treatment (i.e. number of office visits required).

### SUMMARY OF THE DISCLOSURE

The present disclosure uses motion data as an objective facilitator in the behavioral treatment for ADHD to provide a digital therapeutic targeting the behavioral component of ADHD. The method and system of the present disclosure may also be used to provide other interventions and may be implemented in the form of a consumer or other level/range of product.

The method, device and system of the present disclosure provides precision and objectivity using biomarker feedback, such as hyperactivity measure/motion data driven feedback to the patient instead of relying on the subjective observation by the parent/teacher used in the current practice. In accordance with the present disclosure, motion data is used to objectively measure treatment targets (i.e. behaviors) the same way blood pressure is measured to inform treatment decisions.

As seen in the flow diagram of FIG. 4, feedback for the behavior is immediate (through the visual representation/Avatar changes in size/strength or color), consistent and enhanced (where the child is reinforced visually by the Avatar changes, by points earned/accumulated to use for gaming (or other rewards) and by the interactive feature where the parent is able to send the child a reinforcement via app, such as “great job” emoji, for instance while the child is in school and the parent is observing via app that the child is doing well). This addresses the shortcomings of the current practice of using behavioral charts/school report card, leading often to inconsistent and/or delayed feedback which decreases treatment efficacy.

The feedback generated by the present disclosure is nuanced and is based on small incremental improvements in behavior (i.e. behavioral shaping) captured through analysis of motion data using an algorithm. The goal of the present disclosure is for the algorithm to adapt to patient performance and modify the reinforcement as such in order to promote progress (i.e. initially providing reinforcement for any small improvements in behavior but as the child makes progress make it harder to earn rewards in order to maintain a steady challenge). Current practice mainly provides reinforcement based on presence/absence of behavior.

The method, device and system of the present disclosure fosters generalization of treatment effects by integration of the behavioral goals and positive reinforcement for school and home in one place. Current practice is using separate Behavioral Chart at home+Daily Report Card in school which is more time consuming, effort intensive and less effective.

The method, device and system advantageously potentially decreases parental stress by decreasing the parental task burden, namely, to track and reinforce behavior, and possibly increased efficacy of treatment with better outcomes.

As in the present disclosure, there is also an increased opportunity for positive parent child interactions given the parent-child interactive feature and the opportunity for the parent to reinforce the child's positive behavior with more ease given data being provided via app with points earned being calculated. The goal of the method, device and system of the present disclosure is for the points earned to translate into rewards (as predetermined by the parent and previously discussed with the child) or to translate automatically into points or currency to be used in a specific video game which the child could access directly on the app's child interface.

Therefore, the present disclosure increases the cost effectiveness of treatment due to less time spent by the therapist in developing and monitoring treatment where better treatment outcomes result in less need for treatment and fewer office visits. Also, behavioral therapy has often been shown to lower the medication dosage needed when treatment is used in combination with medication. In this context, a behavioral treatment is more effective through the use of the method, device and system of the present disclosure thereby leading to further lowering of medication dosage being used and therefore a lower risk of side effects, which is an outcome that would appeal to parents.

Therefore, an object of the present disclosure is to provide a method, device and system for treating ADHD that is effective, efficient and cost-effective.

A further object is to partially automate and optimize Behavior Parent Training which is a form of Behavior Therapy.

It is a further object to provide a method, device and system that is precise and objective.

A further object is to provide a method, device and system that uses motion data to objectively measure treatment targets.

Yet a further object of the present disclosure is to provide a method, device and system that provides immediate feedback.

Still further, another object of the present disclosure is to provide a method, device and system that fosters generalization by providing reinforcement of behaviors across settings, such as home and school.

Yet another object of the present disclosure is to provide a method, device and system that decreases parental stress in managing a child's ADHD.

Another object is to provide a method, device and system that improves parent-child interactions.

Further, an object of the present disclosure is to reduce the amount of medications given to a child while still treating their ADHD.

Still further, an object is to increase the cost effectiveness of treatment while improving the outcome and reducing the overall need for treatment.

Other objects, features and advantages of the disclosure shall become apparent as the description thereof proceeds when considered in connection with the accompanying illustrative drawings.

### BRIEF DESCRIPTION OF THE DRAWING FIGURES

The novel features that are characteristic of the present disclosure are set forth in the appended claims. However, the preferred embodiments, together with further objects and attendant advantages, will be best understood by reference to the following detailed description taken in connection with the accompanying Figures in which:

FIG. 1 shows an exemplary home token economy program;

FIG. 2 shows an exemplary Daily Report Card;

FIG. 3 shows an exemplary weekly schedule chart as typically maintained for ADHD treatment;

FIG. 4 illustrates the flow of operation of the present disclosure;

FIG. 5 illustrates parent/service provider device interactivity of the present disclosure;

FIG. 6 shows the solution system loop to illustrate how therapists, prescribers and insurance companies utilize the platform to identify, through data analytics, optimized treatment protocols and aid in their population health management;

FIG. 7 is a tiled diagram of the features and interactions of the features of the present disclosure;

FIG. 8 is a schematic diagram illustrating the method and system of the present disclosure;

FIG. 9 shows parent child interaction using the method and system of the present disclosure;

FIG. 10 shows the operation of the device, method and system, as well as the child, in a school setting;

FIG. 11 illustrates the use of an Avatar in the device, method and system of the present disclosure;

FIG. 12 illustrates the reward component of the device, method and system of the present disclosure;

FIG. 13 illustrates the interaction of treatment professionals with the parent using the device, method and system of the present disclosure; and

FIG. 14 illustrates the interaction of the parent with the child using the device, method and system of the present disclosure.

### DETAILED DESCRIPTION OF THE DISCLOSURE

The method, device and system of the present disclosure is shown in detail in the attached drawings figures and the detailed discussion below.

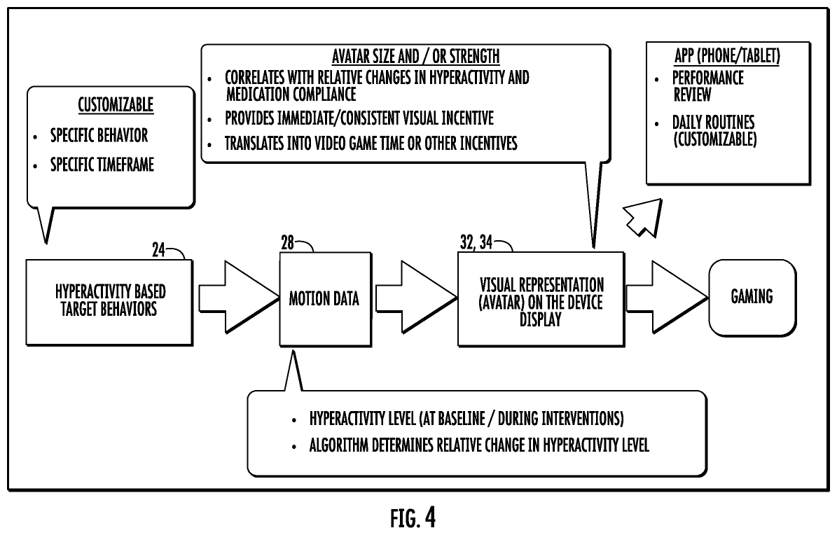

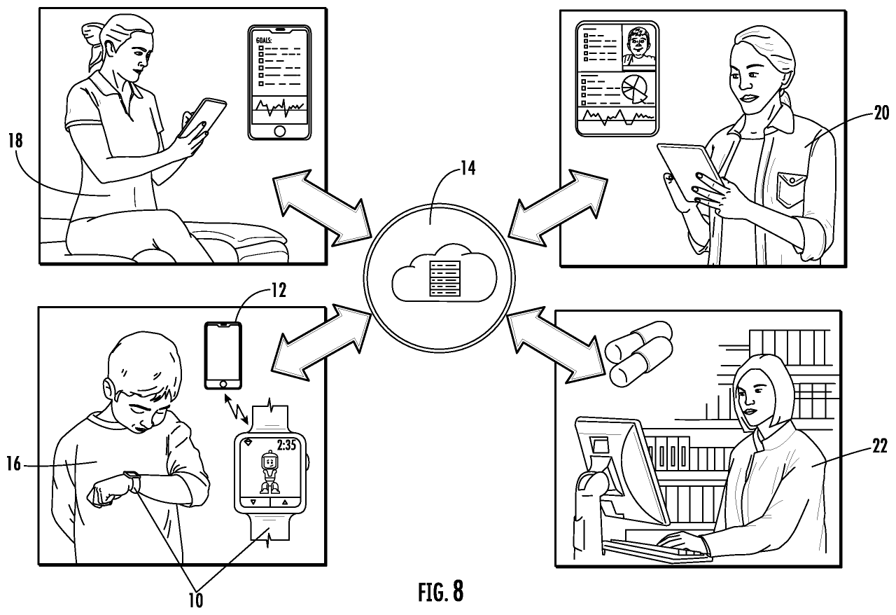

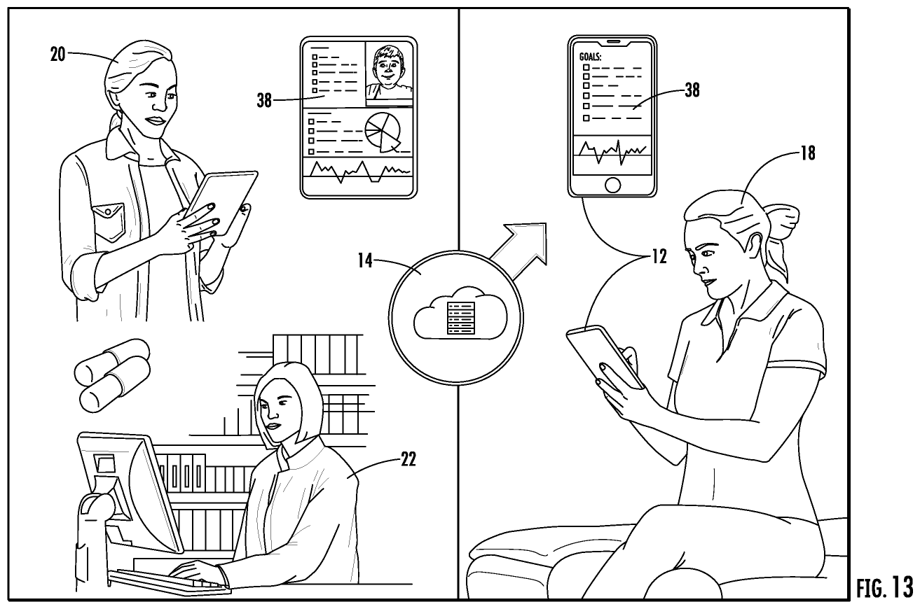

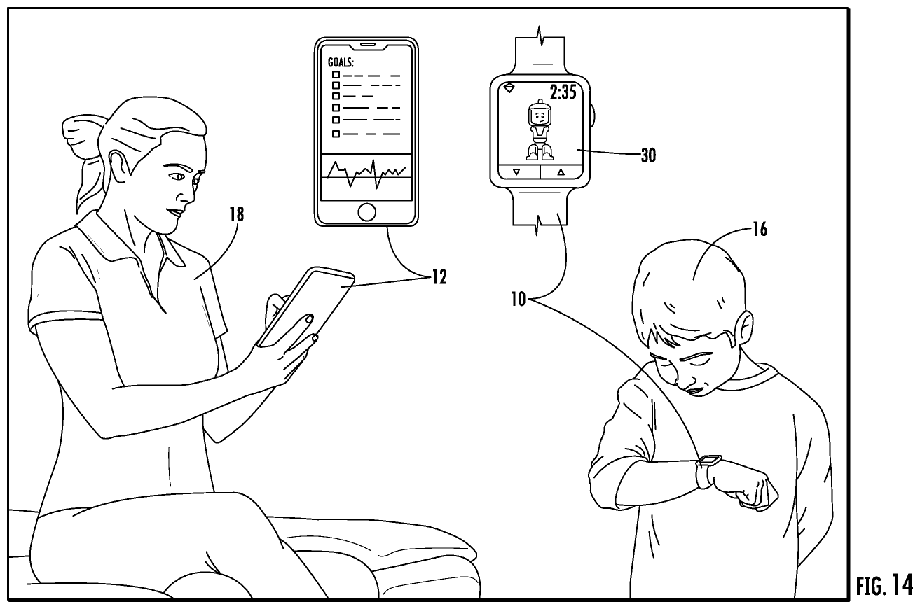

The present disclosure provides a system including a wearable device and using cloud based technology to facilitate the transfer of information allowing parents, therapists and prescribers to track, interpret and implement accurate treatment solutions in real time. The system will be used by a child diagnosed with ADHD. The wearable device in the preferred embodiment is equipped with at least motion sensors. Optionally, the wearable device may also include a location or GPS tracker as a safety feature. Motion data collected by the wearable device is used for two main purposes: 1) to objectively measure hyperactivity levels based on specific metrics (distance, time active, area, micro-events) or other metrics deemed relevant for treatment of ADHD; and 2) to track the child's sleep-wake patterns (given a high percentage of children with ADHD are known to have sleep disorders). Further, the wearable device may detect presence or absence of aggression. Additional sensors may be included to aid the assessment of sleep-wake patterns. Various features of the device, method, and system in accordance with the present disclosure are shown in FIGS. 4-14. The interrelationships of the device, method, and system, as well as the child, parent, therapist and prescriber can be seen illustrated in FIG. 8.

Also, the present invention may employ sensors other than or in addition to motion sensors. For example, heart rate sensors may be used to supplement the amount and type of data collected and increase the sensitivity and accuracy of the present invention.

Turning to FIG. 8 the system of the present disclosure is generally illustrated where the wearable device 10 of the present disclosure is shown as a bracelet but may be any wearable device or tag that is portable and can be kept or worn on the child. The wearable device 10 preferably connects to an electronic device 12 such as a smartphone or tablet having a computer processor therein via a wireless communications connection of any type known in the art. The wearable device interfaces 10 with an app operating on the electronic device 12 and includes both a child and a parent interface. The wearable device 12 also has connectivity to the provider via app connection to an electronic communications network or cloud-based exchange 14, such as both a therapist and prescriber, either through an app or direct integration in the electronic health record through an API. The wearable device is preferably in the form of a watch or a device worn on the wrist. The present disclosure may employ any type of wearable device that can be worn on any part of the body in any form, such as a jacket, vest, shirt, pants hat, shoes, belt, eyewear, and the like. Still further, the wearable device may be in the form of a smartphone or other device that may be held in the hand or stored in a pocket or other location on a person.

The system of the present disclosure as shown at FIG. 8 may have four categories of users, such as child 16, parent/caregiver 18, therapist 20, prescriber 22, and the like, who will interact with the device through different channels and at different points in time with the ultimate goal of the system as a whole to facilitate the delivery of behavioral treatment (i.e. based on contingency theory and involving increasing desired behaviors by providing positive reinforcement) for ADHD. In operation, the child 16 wears the wearable device 10 that detects and collects data from various sensors contained therein. The collected data is transmitted to the app operating on the electronic device 12 for processing and comparison to various behavior targets that are programmed therein. The collected and target compared data is then transmitted from the electronic device 12 via communication network 14 for further interaction and analysis with a parent/caregiver, therapist 20 and or other medical professionals such as prescribers 22, insurance providers, etc., as will be discussed in more detail below.

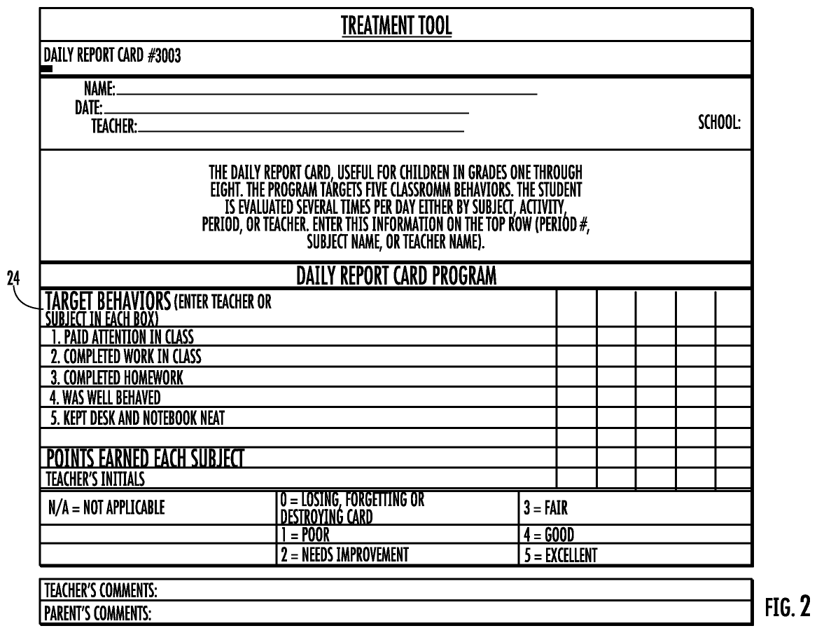

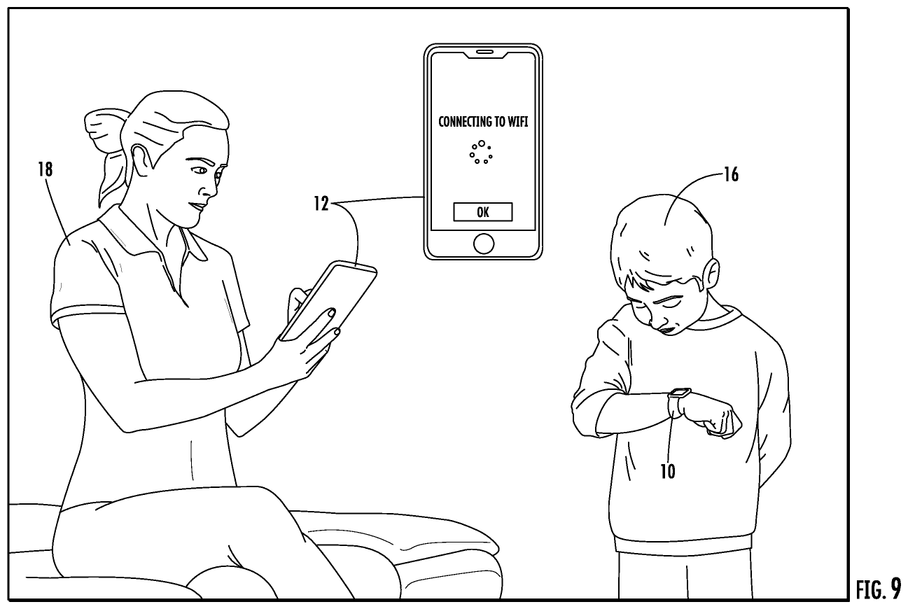

In setting up and maintaining the system of the present disclosure during operation, as shown in FIG. 2, desired target behaviors 24 (up to five total at a given time) are identified by the parent (ideally along with a therapist) as shown in the exemplary daily report card and in the illustration at FIG. 9. The target behaviors 24 are set into the system and then used for baseline comparisons and further processing during the course of treatment. The consideration is to have three motion based behaviors that are selected from a drop-down list in the app (motion based behaviors, such as staying seated during class, no running, sitting still, no aggression) and two non-motion related behaviors (such as medication compliance, complete bedtime routine). The behaviors can be customized to be tracked during specific timeframes, such as school class, dinner time, homework time.

Additionally, the system of the present disclosure may employ a different approach to identifying, tracking and reinforcing child's behavioral functioning in addition to the pre-determined motion based behaviors described above.

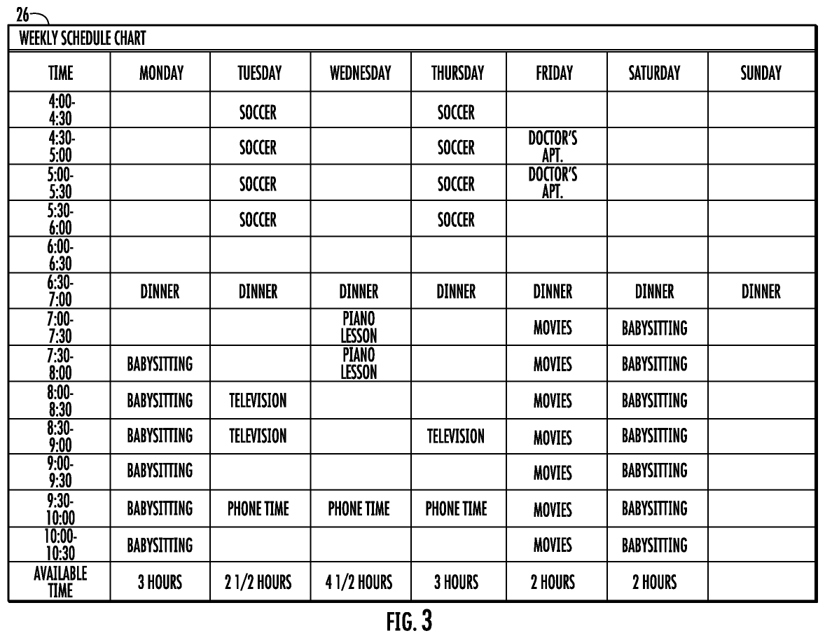

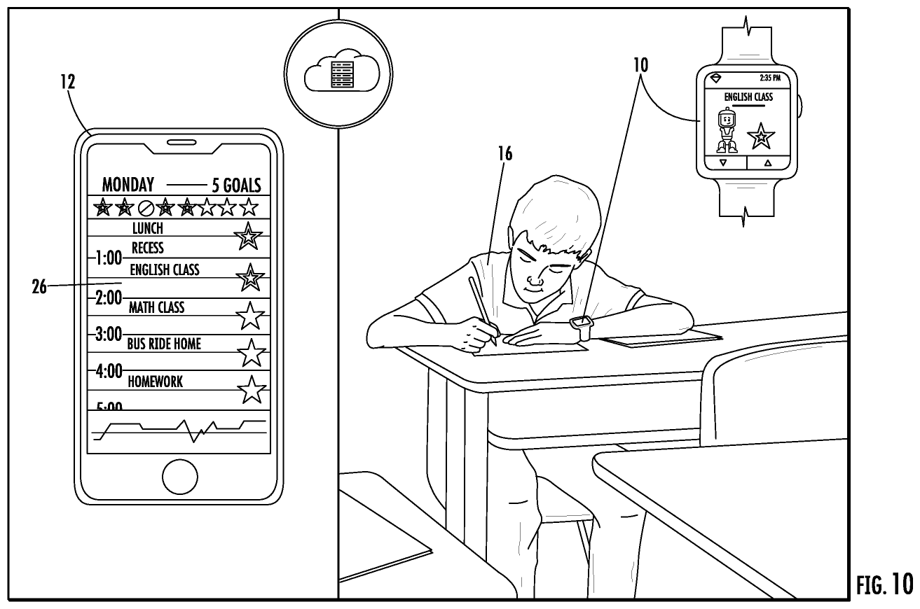

Additionally, in accordance with the disclosure, parents/caregivers preferably create and input routines in the app on the electronic device 12 that generate a schedule 26 for the child 16 as is illustrated at FIGS. 3 and 10 for the child (e.g. morning/bedtime routine) via the connected app (with a parent interface) given that children with ADHD have significant difficulties in this area as part of their executive dysfunction. These routines provide a checklist for the child (including a visual) to follow and therefore aid their successful completion and being a non-motion related behavioral goal that is reinforced as described above.

As shown in the flow charts of FIGS. 4, 5 and 6, motion data 28 is collected and analyzed by an algorithm operating on the electronic device 12 with three main objectives:
1. Determine the child's baseline hyperactivity level (at the beginning of the treatment intervention) based on four specific metrics (distance, time active, area, micro-events) or other metrics deemed relevant. Metrics are preferably used for each specific behavior (e.g. for “no fidgeting”—micro-events/area, for “staying seated during class”—distance, area).
2. Collect motion data 28 as collected in the wearable device 10 and transmit the collected motion data 28 to the electronic device 12 for analysis by the app to determine changes in the hyperactivity level from baseline along those four specific metrics (or other metrics) once the intervention is initiated. The desired behaviors and rewards (positive reinforcement) are reviewed by the parent with the child and the child is aware of the plan/expectations which marks the beginning of the intervention. Also, the desired behaviors and rewards are reviewed with the child on an ongoing basis.
3. Determine based on pre-set parameters at what level of change the rewards will be generated and presented to the child. The algorithm adapts to the child's performance in order to deliver an appropriate challenge (not too difficult or too easy) and foster progress (i.e. behavioral shaping=initially reinforce small changes in behavior and later making it gradually more difficult for the child to earn rewards). For instance, at the start of treatment the reward could be set to be generated for a 10% change (decrease) in hyperactivity level along the specific metrics. However, if after a set period (e.g. 24 hrs.; specific duration to be determined during proof-of-concept studies) there is only a maximum 6% change in hyperactivity the algorithm will adapt and generate rewards for 6%. Subsequently after a set period (e.g. 48 hrs.) of the child consistently achieving 6% change in hyperactivity, the algorithm will start generating rewards for a higher % change (e.g. 7%) in order to increase the level of challenge and foster progress. While an embodiment of the algorithm is set forth below, it should be understood that the algorithm may be modified to suit the application at hand and the particular patient or health issue to be treated, such as ADHD.

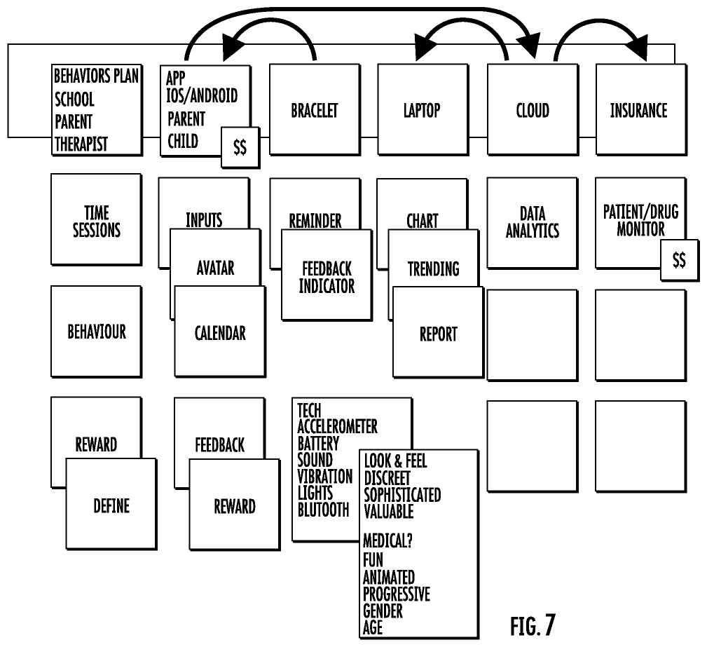

Further, FIG. 7 shows a tiled diagram of the features and interactions of the features of the present disclosure as discussed in detail herein.

To add more nuance to the algorithm of the present disclosure and more intensive re-enforcement, different degrees of rewards (e.g., number of points) for different performance level (% change in hyperactivity metrics) may be rewarded. For instance, a 4% change provides 10 points, and a 6% change provides 20 points.

The pre-set parameters based on which rewards are generated will be contingent on additional factors. The child's age will be one of the contingencies given expected developmental variations of the child's ability for self-control and response to rewards (e.g. younger children may require reinforcements for smaller % changes). Additionally, the algorithm will adapt the parameters (i.e. % change based on which rewards are generated) to the child's sleep quality in a given day reflected by a “sleep score” (determined based on the analysis of sleep data) given the expected possible decline in a child's overall performance in the context of poor sleep. The “sleep score” and specific cut-offs to classify the sleep quality (e.g. normal or mild/moderate/severe sleep impairment) will be determined through proof-of-concept studies. Other contingencies may be considered in algorithm development for example gender, setting (i.e. school or home) among others. Furthermore, the algorithm and specifically the pre-set parameters will be fine-tuned based on population-level performance with growing population data being collected.

The present disclosure also provides additional consideration for utilization of motion data and other physiological data. For example, machine learning tools (e.g. entropy analysis) will be used to assess impulsivity or inattention (the other core features of ADHD besides hyperactivity) using this wearable. Additionally, similar data analytics tools may be employed to make a determination relative to the presence or absence of aggression which could be part of the ADHD clinical picture. The ability to detect/track those symptoms further strengthens the therapeutic value of the wearable device as they could be included among the target behaviors (e.g. lack of aggression) to be tracked and reinforced.

The method, device and system of the present disclosure provides for child device interaction. The child 16 wears the wearable device 10 continuously (most of the day and night and across settings) and will be able to access the app (using a child interface) operating on the electronic device 12 and connected to the wearable device 10 on a smartphone or tablet.

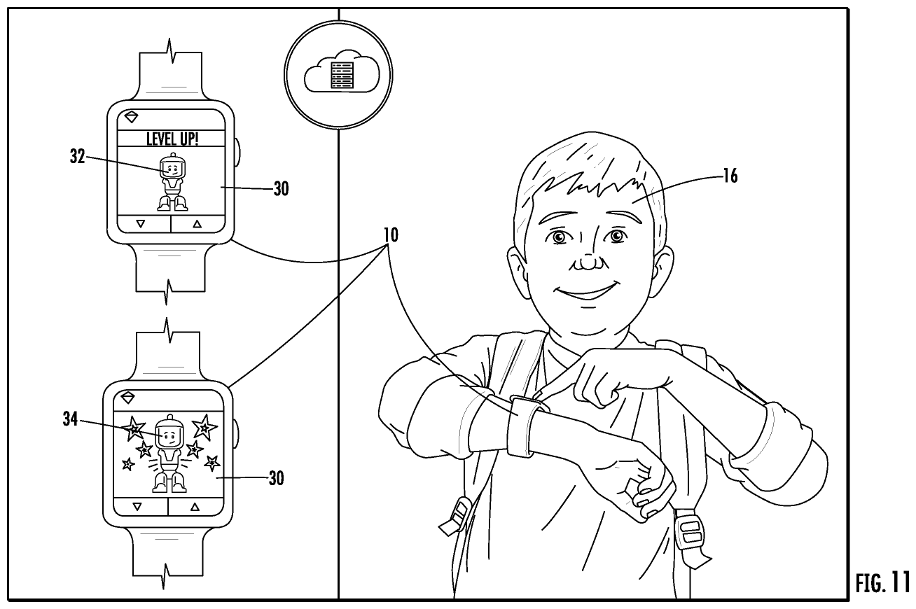

As described at FIG. 4 and illustrated at FIG. 11, the wearable device 10 includes a display 30 on which the child 16 chooses an Avatar 32 (from several alternatives provided) to be displayed and used as one of the channels for rewards delivery. The Avatar's size/color or strength changes as shown in improved Avatar 34 in correlation with the rewards/points determined by the algorithm. Several Avatar choices are provided to appeal to children of different age, gender, cultural background. The app interface operating on the electronic device 12 presents the list of desired behaviors (up to five), the daily routines (as entered by parents) and a performance review/points balance for viewing by the child 16. An interactive feature of the wearable device 10 prompts the child visually or haptically through a display or vibration each time the tracking period is starting (e.g. math class is starting and he/she is expected to sit in the chair) and may provide reminders of target behaviors in a child friendly format.

The wearable device 10 itself, includes a display 30 where the child 16 can see the Avatar 32 and its changes to an improved Avatar 34. The changes in the Avatar constitute the visual component of the positive reinforcement that is updated immediately following the desired behavior and the Avatar update is delivered autonomously without requiring caregiver firsthand involvement.

Additionally, the system of the present disclosure may employ a different approach for the visual component of the positive reinforcement other than Avatar if deemed necessary in order to optimize its impact and attend to a diverse patient population.

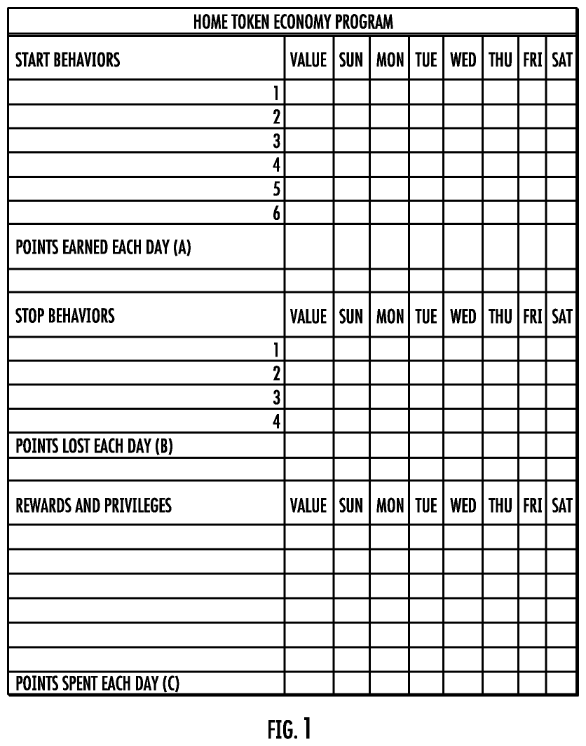

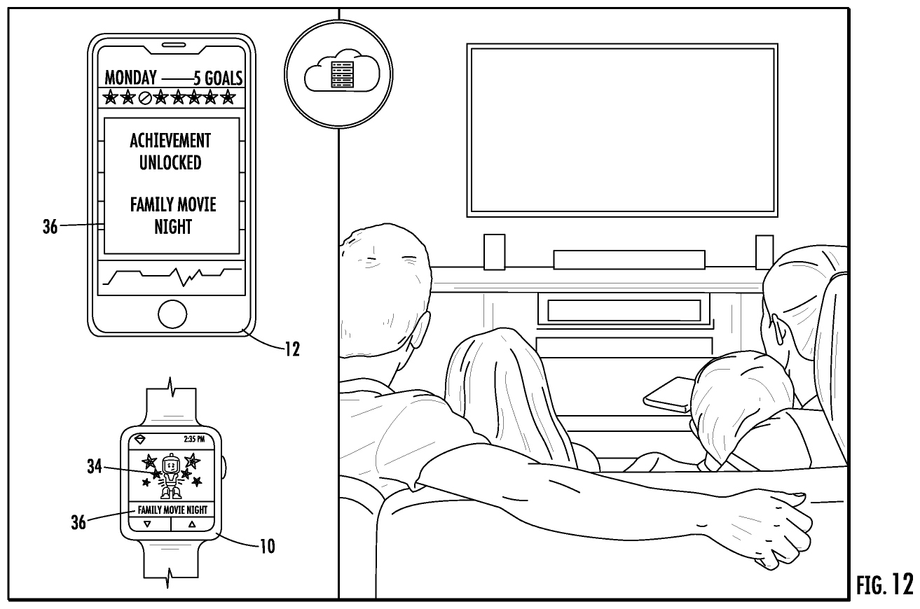

The positive reinforcement for the desired behavior has two additional components. The first one entails points earned as calculated by the algorithm. Further points can be entered manually by the parent for the non-motion related goals. The points will be presented as illustrated in FIGS. 1 and 9 to the child 16 using the app in a summary format at the end of each day/week/month as points balance and performance review in form of a graph or other child friendly visual. As shown at FIG. 12, the points are then translated into a reward 36 such as family movie night, videogame time or other rewards as predetermined by the parent 18 and previously discussed with the child 16. Ultimately the goal is for the points to translate automatically in videogame time or features in a particular game that the child accesses or plays through the app interface.

The second component stems from the parent being able to see in real-time the child's performance (via app interface) and through an interactive feature to be able to send a random reinforcement to the child, such as a “great job” emoji for presentation to the child 16 on the display 30.

The app interface operating on the electronic device 12 presents to the child 16 the expected daily routines (e.g. morning and night) in form a picture board or another child friendly format.

The wearable device 10, is connected to the app operating on the electronic device 12 and also may be connected via the electronic device 12 and communications network 14 and integrate with software platforms used by teachers to communicate homework schedule/requirements (e.g. eChalk). This will assist children having ADHD with their planning challenges as part of their executive dysfunction. Additional features in some embodiments may include the device prompting the child to engage in up to 30 min of moderate-vigorous physical activity daily given data showing acute physical exercise to lead to improvement in executive functioning in children with ADHD. This could be a set goal for the child with its completion being determined by tracking motion data. Further, integration with exergaming can facilitate the child's engagement in a moderate-vigorous physical activity.

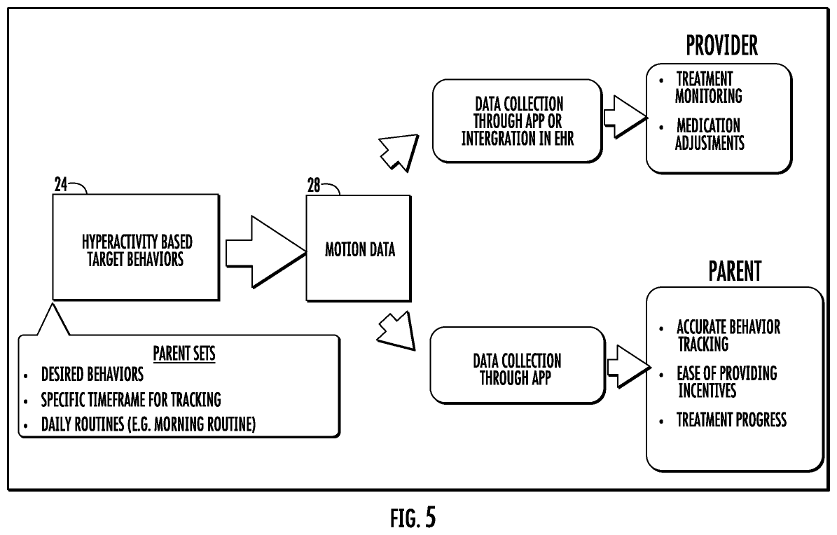

The method, device and system of the present disclosure also provides a unique parent-device interactivity, as seen in FIGS. 5 and 12. For the example, the parent will interact with the device primarily through the app interface which will allow her/him to enter behavioral goals and daily routines, enter points for the non-motion based goals (e.g. medication compliance/routine completion) and see child performance. The parent will identify desired behaviors, ideally along with a therapist, and enter them through the app. Additionally, the parent will create the daily routines by inputting them through the app from a drop-down list, such as get out of bed, brush teeth, get dressed, or the like.

The parent will be able to see the child's performance on the motion based goals both in real time and at the end of the day/week/month. In this context, the parent will be able to reinforce the child both in real time through the interactive feature (e.g. sending “great job” emoji) and at the end of the day/week/month. The parent will input manually points for the non-motion based goals (e.g. medication compliance).

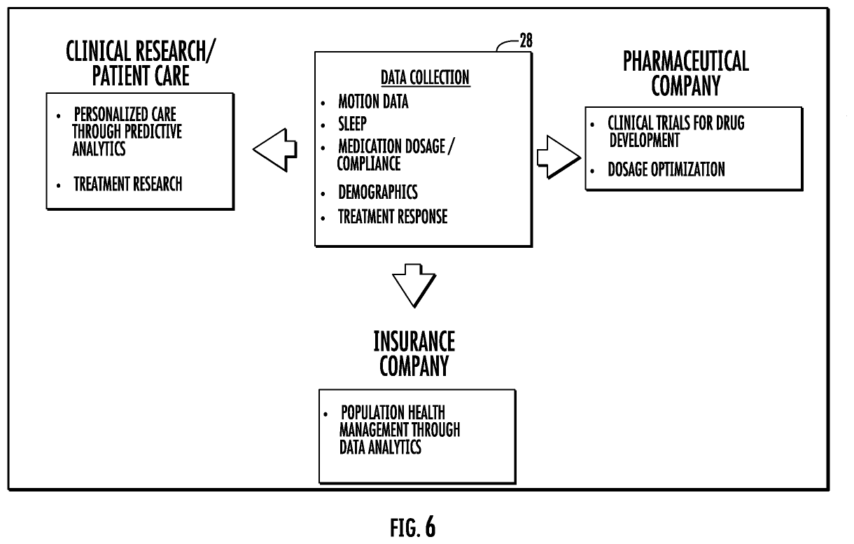

Therapist 20 and prescriber 22 interaction is also provided with the present disclosure, as seen in FIGS. 6, 8 and 13. For example, the therapist 20 and prescriber 22 interacts with the device mainly through the wireless communication network 14 directly to the app operating on the electronic device 12 or through the electronic medical record which is integrated with the app. The interface shares a child's performance, analyzed motion data (e.g. graphs showing hyperactivity variation throughout the day), and sleep data, which would inform adjusted treatment decisions, for prescriber (medication adjustments) and for therapist behavioral goals adjustments. These changes in treatment 38 are sent by the medical professionals for presentation on the parent interface of the app operating on the electronic device 12 or any other electronic device carried by the parent.

Other users may interact with the method and system of the present disclosure. The growing customer base coupled with the long-term use of the wearable device will allow for de-identified and privacy compliant data collection including motion and sleep data (and other sensor data), medication choices/dosage/compliance, treatment response and demographics. The accumulated data provides a valuable platform for multiple uses such as 1) Clinical/treatment research including but not limited to the use of predictive analytics to identify determinants of treatment response or risk factors for treatment resistance; and 2) pharmaceutical companies could utilize this platform and the device to aid their clinical trials for drug development including dosage optimization. Currently, drug efficacy during clinical trials is determined by using questionnaires administered either by a clinical investigator (e.g. ADHD Rating Scale (ADHD-RS), Clinical Global Impression-Improvement (CGI-I) rating scale) or by the parent (Conners' Parent Rating Scale). While those questionnaires are standardized tools, they add a level of subjectivity and are also labor/staff intensive therefore costly. For instance, ADHD Rating Scale (ADHD-RS) is an 18-item questionnaire with a score range of 0-54 points that measures the core symptoms of ADHD which includes both hyperactive/impulsive and inattentive subscales, and 3) Insurance companies could also utilize the platform to identify through data analytics optimized treatment protocols and aid in their population health management, as in FIG. 6.

It should be understood that the invention herein is not limited to the treatment of ADHD. The same principles, tools, methods, devices and system of the present invention can be used for treating other health issues, such as other disruptive behavior disorders, obesity, depression, and the like.

While there is shown and described herein certain specific structure embodying the disclosure, it will be manifest to those skilled in the art that various modifications and rearrangements of the parts may be made without departing from the spirit and scope of the underlying inventive concept and that the same is not limited to the particular forms herein shown and described except insofar as indicated by the scope of the appended claims.

What is claimed is:

1. A method for the treatment of ADHD, comprising the steps of:
providing a wearable device, said wearable device being connectable to an electronic device having an app thereon;
defining a plurality of desired behaviors within the app, said behaviors including a plurality of motion based behaviors;
defining and creating a plurality of input routines within said app;
defining and creating pre-set parameters for generation and presentation of rewards on the wearable device;
collecting motion data and/or location data via sensors in the wearable device;
analyzing the motion data in the electronic device by an algorithm, comprising the steps of:
determining a baseline compliance with the behaviors based on the analyzed motion data;
determining deviations from the baseline compliance with the behaviors and routines;
comparing the deviations to said pre-set parameters; and
presenting a reward based on a positive determination of the deviations.

2. The method of claim 1, further comprising the step of:
assigning different levels of deviation required for presentation of the reward.

3. The method of claim 1, further comprising the step of:
transmitting the analyzed motion data to a predetermined group of treatment providers.

4. The method of claim 3, wherein the group of treatment providers are selected from the group consisting of: parents, therapists, prescribers, teachers and insurance providers.

5. The method of claim 1, further comprising the step of:
reviewing the analyzed motion data over time to determine a compliance level with the input routine and the desired behaviors.

6. The method of claim 5, further comprising:
adapting the pre-set parameters based on the review.

7. The method of claim 1, wherein the wearable device includes a display screen to display a pre-selected avatar.

8. The method of claim 7, wherein an appearance of the avatar changes based on the deviations.

9. The method of claim 7, wherein a parent/caregiver views the analysis of motion data in real time and interactively present additional rewards for display on the display screen.

10. A system for the treatment of ADHD, comprising:
a wearable device, the wearable device being connectable to an electronic device having an app thereon;
the app containing a plurality of desired behaviors including a plurality of motion based behaviors, a plurality of input routines, and pre-set parameters for generation and presentation of rewards on the wearable device;
wherein the app collects motion data and/or location data via sensors in the wearable device and analyzes the motion data in the electronic device by an algorithm, comprising the steps of:
determining a baseline compliance with the behaviors based on the analyzed motion data;
determining deviations from the baseline compliance with said behaviors and routines;
comparing said deviations to the pre-set parameters; and
a display on the wearable device for presenting a reward based on a positive determination of the deviations.

11. The system of claim 10, further comprising:
the app assigning different levels of deviation required for presentation of the reward.

12. The system of claim 10, further comprising:
the electronic device transmitting the analyzed motion data to a predetermined group of treatment providers.

13. The system of claim 12, wherein the group of treatment providers are selected from the group consisting of: parents, therapists, prescribers, teachers and insurance providers.

14. The system of claim 10, wherein analyzed motion data is reviewed over time to determine a compliance level with the input routine and said desired behaviors.

15. The system of claim 14, wherein the pre-set parameters are adapted based on said review.

16. The system of claim 10, wherein the display screen displays a pre-selected avatar.

17. The system of claim 16, wherein an appearance of the avatar changes based on the deviations.

18. The system of claim 10, wherein a parent/caregiver views the analysis of motion data in real time and interactively present additional rewards for display on the display screen.

19. The system of claim 10, wherein a provider (therapist or prescriber) views the analysis of motion data and communicates adjustments in treatment to the parent/caregiver.
